(19) United States
(12) Patent Application Publication
(10) Pub. No.: US 2011/0217260 A1
(43) Pub. Date: Sep. 8, 2011

(54) METHOD OF ENHANCING EYELASH AND EYE BROW HAIR GROWTH

(76) Inventors: Totada R. Shantha, McDonough, GA (US); Erica Maya Shantha, McDonough, GA (US); Jessica Gowramma Shantha, McDonough, GA (US); Usha T. Shantha Martin, Macon, GA (US); Lauren Asha Shantha, McDonough, GA (US)

(21) Appl. No.: 12/717,746

(22) Filed: Mar. 4, 2010

(51) Int. Cl. A61K 8/64; A61Q 7/00; A61K 8/28; A61K 38/20; A61K 38/13; A61K 36/48

(52) U.S. Cl. 424/85.2; 424/757; 514/6.5; 514/8.1; 514/8.7; 514/8.9; 514/20.7; 424/70.14

(57) ABSTRACT

A method of enhancing eyelash and eye brow hair growth utilizes insulin and/or IFG-1 alone or in combination with other known hair growth promoting therapeutic, pharmaceutical, biochemical and biological agents to increase the effect of various therapeutic hair regrowth strategies to enhance eyelash and eye brow hair growth.

### BACKGROUND OF THE INVENTION

Dermatologists, cosmetologists, and hair dressers recognize many types of eye lash and eye brow hair loss, and come across woman especially seeking longer luscious eye lashes to add to their makeup beauty. To understand the eye lashes growth, it is important to have knowledge about the hair histology, physiology and the factors contributing to their growth or loss.

The hairs are found all over the skin surface of the human body. Hair is a keratinized dead cells derived from the multiplying epithelial cells situated at the base of the hair follicle as matrix. Hairs are absent from the palms of the hands, the soles of the feet, the dorsal surfaces of the distal phalanges, the umbilicus, the glans penis, the inner surface of the prepuce, the inner surfaces of the clitoris, labia majora and minora, conjunctiva along with cornea. The hairs differ in length, thickness and color in different parts of the body. Hairs differ in different races of human and sex. Hair in the skin of the eyelid and brows has a short projection for a short distance. The scalp hairs are long as compared to the eyelashes.

A hair consists of a root, the part implanted in the skin, and a shaft (scapus), the portion projecting from the surface. The root (radix) of the hair has a proximal enlargement called hair follicle embedded in the bulb, which is set in an invagination of the epidermis and superficial portion of the corium, called the hair follicle. A hair follicle is a tiny cup-shaped pit buried deep in the fat of the scalp. The follicle is the point from which the hair grows. It is richly supplied with blood vessels and surrounded by delicate sensory nerve fibers. The temperature around the follicle is normal body temperature, and is not easily affected by cold or hot weather. The hair follicle can be divided into two regions.

1. The hair bulb lies inside the hair follicle. It is a structure of actively growing cells, which eventually produce the long fine cylinder of a hair.

2. The portion of hair we all desire and notice is protruding above the level of the epidermis is called the hair shaft, and the portion within the follicle buried below the epidermis is the hair root or bulb (see FIGS. 1 to 6).

When the hair is of considerable length, the follicle extends into the subcutaneous tissue. The longer the eye lashes, the deeper the follicle extends. Compared to scalp, beard and pubic hairs, the follicle is superficially placed, hence eye lashes cans be pulled out without much effort and pain. The hair follicle commences on the surface of the skin with a funnel-shaped opening, and passes inwards and become dilated at its deep extremity, where it corresponds with the hair bulb which gets incorporated into hair follicle. The ducts of one or more sebaceous glands open into the follicle near the skin surface. At the bottom of each hair follicle there is a small conical vascular prominence called dermal hair papilla which is continuous with the dermal layer of the follicle, and is supplied with myelinated and non-myelinated nerve endings. The hair derives its nutrition for its growth from the capillaries in the papilla and trophic factors from the sensory nerves.

Much is known about various types of human hair and its growth patterns on various parts of the body. Growth of a hair occurs at the hair bulb due to proliferation of the cells capping the papilla and form the germinal matrix of the hair. The duration of life of a single hair varies from about four months (eyelashes, eye brows) to about four to seven years (scalp hair, and adult beard), after which it is shed and is replaced by the sprouting of new cells from the germinal matrix (anagen phase) from the follicle after a period of rest (Telogen phase).

The hair follicle has a complex histological structure. It is composed of epithelial and connective tissue sheaths. The epithelial sheath is in close contact with the hair root and is made up of two layers; inner and outer. The inner layer is composed of three sub layers of cells: (a) an inner layer, the cuticle, which is similar and in close contact with the hair cuticle; (b) a middle Huxley's layer, made of a few rows of square cells; and (c) an outer, Henle's layer, made of one row of polygonal, flattened cells. The outer epithelial layer is considered to be nothing but down growth of epidermis, with the spinous layer inside and the basal layer and basal lamina outside. The basal lamina is thickened and known as the vitreous membrane. A connective tissue sheath is an extension of the dermis: it has two layers, inner papillary and outer reticular. Below the dense connective tissue, and external root sheath lays a glossy membrane (see the FIGS. 2 to 6).

The bottom of the hair root is enlarged and made of cells with high potential for division and differentiation similar to stem cells (see FIG. 2). These cells comprise what is known as the hair matrix. The hair matrix cells divide and move up the follicle, differentiating into either hair cells or inner epithelial sheath cells. Among matrix stem cells there are melanocytes producing pigment of the hair (FIGS. 2-6). The pigment is synthesized from the amino acid tyrosine (catalyzed by the enzyme phenol-oxydase) and transformed through DOPA to dopaquinon.

Further transformation of dopaquinon proceeds in two directions: either spontaneous transformation to indolquinon or through the addition of the amino acid cystein. Polymerization of indolquinon produces the dark pigment, melanin. Polymerization of indolquinon and dopaquinon with added cystein produces the yellow pigment, pheomelanin. Matrix cells during their differentiation ingest (by phagocytosis) melanin or pheomelanin from dendritic elongations of melanocytes.

This is how hair assumes its color: black if melanin is dominant, and yellow or red if pheomelanin is the major pigment. The portion of connective tissue root sheath that is in intimate contact with the hair matrix is known as the dermal papilla. It has a major regulating role in hair growth. (Slobodan M. Jankovic and Snezana V. Jankovic (1998). The control of hair growth Dermatology Online Journal 4(1): 2) Center for clinical and experimental pharmacology Clinical Hospital Centre, Kragujevac, Serbia, Yugoslavia).

There are three types of hair on the human skin besides lanugo hair before birth. They are terminal hairs (scalp, genital and beard) which are dark, thick and long; vellus hairs (rest of the body surface); and modified terminal hairs, such as eye lashes, eye brow and axillary hair. Terminal hairs are coarse, pigmented long in which the bulb of the hair follicle is seated deep in the dermis, hence difficult to pull out. Vellus hairs are fine, short, thin, non-pigmented or lightly pigmented hairs in which the hair bulb is located superficially in the dermis, lack medulla and easy to pluck with least pain.

As alopecia (hair loss) progresses, a transition takes place in the area of approaching hair loss wherein the hairs change from the terminal or modified terminal to the vellus. The function of the hair growth-promoter therapeutic agents is to replace these vellus hair with terminal hair by stimulating the growth in the hair follicle and bulb. The same principle also applies for modified terminal hairs such as eye lashes.

Hair Growth

In humans, hairs grow in cycles which are not synchronized. Each hair undergoes 3 phases of the growth cycle at a different time namely: anagen, catagen and telogen (K. S. Stenn and R. Paus (2001). “Controls of Hair Follicle Cycling”. Physiological Reviews 81 (1): 449-49.). Anagen is the phase of active hair growth—approximately 90% of all hairs are in anagen lasting 2 to 7 years, depending on skin region. After anagen is completed, the hair enters catagen; during this short phase (2-3 weeks) the matrix cells gradually stop dividing and eventually keratinize.

When full keratinization is achieved, the hair enters the last phase of the cycle, telogen phase lasting 3-4 months. The keratinized hair falls out. After this telogen phase, new matrix is gradually formed from stem cells in basal layer of outer epithelial root sheath bulge and germinal cells surrounding the papilla. A new hair starts to grow and the follicle is back in anagen phase. In summary, the hairs undergo the following growth and shedding cycles:

Anagen—active growth—new hair is pushing out the old fiber and the follicle is growing deep for nourishment.

Catagen—the transitional phase. Hair detaches from the blood supply and the hair follicle shrinks.

Telogen—Resting phase. Hair fiber easily pulls out.

Mesanagen—a returning to growth

The testosterone, an androgenic hormone stimulates hair growth when applied topically to the deltoid area and injected into the beard and pubic regions. Oral administration result in an increased hair growth in the beard, pubic region, on the trunk and extremities. It is ineffective when applied topically to the scalp balding area and may even case more thinning of the hair. Eye lashes are similar to scalp hairs; androgen application to the eye lashes will have no effect on their growth.

Androgens have diverse effects on hair growth in different body regions (Randall V A, Thorton M J, Hamada K, Messenger A G. Androgen action in cultured dermal papilla cells from human hair follicles. Skin Pharmacol 1994; 7: 20-6). Effects vary from essentially nonexistent (e.g. on eye-lashes), weak (on temporal and sub occipital region hair), moderate (on extremity hair), or strong (on facial, parietal region, pubic, chest, and axillary hair).

Androgens bind to receptors both in the cytoplasm and nuclei of dermal papilla cells and stem cells of the sheaths of the follicle, but only if the hair is in anagen or telogen phase (Sawaya M E. Purification of androgen receptors in human sebocytes and hair. J Invest Dermatol 1992; 98(6 Suppl):925-965. Chondrhry R, Hodgins M B, Van-der Kwast T H, et al. Localization of androgen receptors in human skin by immunohistochemistry: implications for the hormonal regulation of hair growth, sebaceous glands and sweat glands. J Endocrinol 1992; 133: 467-75).

Among all androgens, dermal papilla cells are most affected by 5α-dihydrotestosterone (5α-DHT). It is synthesized in these cells from testosterone under catalytic action of the enzyme 5 α-reductase (Randall V A. Role of 5α-reductase in health and disease. Bailliers Clin Endocrinol Metab 1994; 8: 405-31). This enzyme exists in two forms (isoenzymes)—type I and type II (Horton R. Dihydrotestosterone is a peripheral paracrine hormone. J Androl 1992; 13: 23-7). 5α-dihydrotestosterone is further reduced to 3α-androstanediol which, after conjugation with glucuronic acid, is excreted in urine.

Plasma and urine levels of 3α-androstanediol glucuronide are precise clinical indicators of the extent of testosterone transformation to 5α-DHT (Toscano V, Balducci R, Bianchi P. et al. Two different pathogenetic mechanisms may play a role in acne and in hirsutism. Clin Endocrinol Oxf 1993; 39: 551-6). They are elevated in hirsute women.

Another important enzyme that plays a role in hair loss is Aromatase, which present in the skin fibroblasts and absent in the dermal papilla. Aromatase activity has been detected in plucked human scalp hair follicles (Schweikert H U, Milewich L, Wilson J D: Aromatization of androstenedione by isolated human hairs. J Clin Endocrinol Mtab 40: 413, 1975. Sawawa M E, Penneys N S: Immunohistochemical distribution of aromatase and 3B-hydroxysteroid dehydrogenase in human hair follicle and sebaceous gland. J Cutan Path01 19: 309, 1991). In hair follicles in most body sites, testosterone acts as a pre-hormone and the response to testosterone is determined by which metabolic pathway predominates as disclosed in U.S. Pat. No. 6,020,327.

In skin sites where 5α-reductase activity is high, such as in the beard, testosterone is metabolized to DHT which has a stimulatory action on hair growth on the rest of the body except the scalp. In scalp, testosterone is metabolized mainly via the aromatase pathway to estrogens causing inhibition of hair growth, hence balding and hair thinning. Aromatase activity is under receptor-mediated androgenic control; hence balding does not occur in testicular feminization.

The greater severity of balding in men compared with women can be explained by higher levels of estrogens being formed in the male scalp due to androgen stimulated aromatase activity. It is known that the estrogens inhibit the hair growth in mammals (Moho M P: The effects of different hormonal states on the growth of hair in rats, in Montagna W, Ellis R A (eds): The Biology of Hair Growth, New York, Academic Press, pp 335-398, 1958).

An analogous situation exists in the brain where some characteristics of male behaviors in a number of avian and mammalian species are determined by estrogens synthesized locally by androgen-dependent aromatization of androgens (Hutchinson J B: Aromatase: neuromodulator in the control of behavior. J Steroid Biochem Molec Biol 44: 509. 1993). This hypothesis is also able to encompass a role for 5α-reductase; as DHT is a more potent androgen than testosterone, circulating or locally formed DHT can be a more effective inducer of aromatase. Interestingly, there are no studies made on the aromatase pathway in the eye lashes, eye brows, and axillary hairs. As they are terminal hair like scalp and beard hair, hairs in the eye lashes are either under the influence of DHT and/or aromatase pathway or neither or both.

Dexamethasone stimulated aromatase 10-20 fold in fibroblasts and induced aromatase in dermal papilla cell. The high levels of aromatase in fibroblasts from frontal scalp are consistent with hypothesis that it plays a role in frontal male baldness. The recognized inhibitory effect of estrogens on hair growth would need to derive from the surrounding dermis as aromatase activity is absent in dermal papilla cells. There is no evidence that androgens act directly on the hair follicle in the balding process. The alteration in skin texture of subjects of a balding scalp suggests that there is an alteration the skin structure which may play a role in the hair loss or hair growth inhibition besides the reduced blood, nutrition and oxygen supply to the area of balding. Hence, it is likely that the inhibition of hair growth is a secondary response to more general changes in local biology of the skin.

U.S. Pat. No. 6,020,327 discloses many factors involved in the hair growth including aromatase inhibitors which are incorporated in the following description. There is great evidence that androgen hormones are necessary for the development of balding; though the mode of hormone action on the hair follicle is unspecified. At present there is no satisfactory explanation and treatment for scalp hair loss (balding). Beard and pubic hair need androgens to prolong the anagen phase for hair growth as exemplified by the appearance of the beard with elevated testosterones in male teenagers. Scalp hair does not need androgens for hair growth but are needed for balding.

It is important to note that there is no difference between the balding and non balding male in their plasma concentration to testosterone. It is thought that the in the skin sites where 5α-reductase activity is high, such as in the beard, testosterone is metabolized to DHT which has a stimulating action on hair growth. On the other hand, in the scalp, the testosterone is metabolized mainly via the aromatase pathway to estrogens causing inhibition of hair growth hence balding effect.

The greater seriousness of balding in men compared with women is explained by higher levels of oestrogen being attained in the male scalp by virtue of androgen stimulated aromatase activity pathway. The formed DHT can be a more effective inducer of aromatase cycle as DHT is a more potent androgen than testosterone. The circulation or locally formed DHT can be a more effective inducer of aromatase resulting is female hormone formation resulting in male pattern baldness. Administration of 5α-reductase inhibitors and Aromatase inhibitors in macaque's monkeys which undergoes androgen dependant scalp hair loss like human prevents and reverses the hair loss. Men with type II 5α reductase deficiency do not go bald either supporting the hypothesis it plays in converting testosterone to DHT which stimulates the aromatase inhibitors to form estrogens. This is another indication of the important role played by 5α-reductase and aromatase in balding of male.

The Eyelids: Anatomy and Histology (FIGS. 1 to 6)

Understanding of the eye lid structure is very important to understand the how our invention works to enhance the eye lashes growth and where to apply the therapeutic agents (see FIG. 8). The eyebrows are two arched eminences of skin, which surmount the orbits and support numerous short, thick hairs modified terminal hair directed obliquely on the surface towards the periphery of the forehead. Muscle fibres of the orbicular oculi, corrugator and frontal belly of the occipitofrontalis are inserted into the skin of the eyebrows imparting various expressive movements to the eye brows depending upon the emotional status of the individual (see FIGS. 1 and 5).

The eyelids protect, nourish and sustain health of the cornea (FIGS. 1-6). The basic structure is divided arbitrarily by surgeons into 2 lamellae, anterior and posterior. From without inwards, each eyelid consists of: skin, subcutaneous areolar tissue, fibres of the orbicular oculi, tarsus plates and orbital septum, tarsal glands and conjunctiva. The upper eyelid contains, in addition, the aponeurosis of the levator palpebrae superioris (see FIGS. 1, 3 and 5). The skin is exceptionally thin and continuous at the margins of the eyelids with the conjunctiva (see FIG. 1). The subcutaneous areolar tissue is lax, delicate, and seldom contains any adipose tissue. That is why in obese people, the fat in the eye lids is still sparse compared to the rest of the body.

The upper eyelid is the larger and more movable, and is furnished the levator palpebrae superioris (FIGS. 1, 5). The two eyelids are united to each other at their ends (FIG. 8), and when the eye is open an elliptical space, termed the palpebral fissure, their margins join at the angles (canthus) of the eyes are noted. The eyelids or palpebrae are thin, movable folds, tailored to fit the front of the eye ball (FIGS. 3, 4), protecting it by their closure, from injury and insult by foreign bodies. The lateral angle of the eye (lateral canthus) is more acute than the medial, and lies in close contact with the eyeball. The medial angle (medial canthus) is prolonged for a short distance towards the nose, and is about 0.6 mm away from the eyeball; the two eyelids are here separated by a triangular space, named the lacus lacrimalis, in which a small reddish body, termed the caruncula lacrimalis, is situated. On the margin of each eyelid, at the basal angles of the lacus lacrimalis, there is a small conical elevation, termed the lacrimal papilla, the apex of which is pierced by the beginning of the lacrimal canaliculus known as the punctum lacrimale which drains the excess of lacrimal secretions conducting to the nasal cavity (FIG. 8).

Each eyelid are covered on its anterior surface with delicate skin; this contains the follicles of some very fine hairs (vellus), sebaceous and sweat glands; and the edges are lined 3-4 rows of with modified short thick dart terminal hairs called eye lashes. The dermis is a loose texture, and the subcutaneous tissue, deep to it contains almost no fat. The keratin of the epidermis gradually thins out as the skin approaches the free margin of the eyelids, and here the epidermis becomes continuous with the epithelium of the palpebral conjunctiva lining the inner (posterior) side of the lid and the eye ball (FIG. 1, 3, 4).

Each lid is reinforced with thin elongated, dense connective tissue tarsal plates (FIGS. 1, 5). They are about 2.5 cm long; one is placed in each eyelid and contributes to its form and support. The tarsal plates are placed in the posterior part of the lid so that the palpebral conjunctiva is opposed to its posterior surface. The secretory portions of long, vertically disposed, complex sebaceous glands, called meibomian glands, are embedded in the tarsal plate. They open onto the posterior part of the free margin of the lid behind the eye lashes (FIGS. 1.3-6). They are modified sudoriferous glands also termed ciliary glands, are arranged in several rows close to the free margin of each lid and open behind the attachments of the eyelashes.

The upper lid has more Meibomian glands compared to the lower lids, which may account for the greater incidence of sebaceous carcinoma in the upper eyelids. If one of these glands becomes infected, a painful small pea-like swelling develops in the lid.

The inner lining of the eyelids is conjunctival epithelium, also contains a many goblet cells, located more at the formix and less at the margin of the lid. The tarsus of the upper eyelid is a semi-oval form, about 10 mm in height at the centre, and gradually narrowing towards its extremities. The lowest fibres of the superficial lamella of the aponeurosis of the levator palpebrae superioris are attached to its anterior surface, and the deep lamella of the same aponeurosis is inserted into its upper margin. The tarsus of the lower eyelid is a narrower and the vertical diameter is about 5 mm. The free or ciliary margins of the tarsi are thick and straight. The attached or orbital margins are connected to the circumference of the orbit by the orbital septum. The lateral ends of the tarsi are attached by a band, named the lateral palpebral ligament, to a tubercle on the zygomatic bone, just within the orbital margin. This ligament is separated from the more superficially placed lateral palpebral raphe by a few lobules of the lacrimal gland. The medial ends of the tarsi are attached by a strong tendinous medial palpebral ligament to the upper part of the lacrimal crest, and to the adjoining part of the frontal process of the maxilla.

The orbital septum is a weak membranous sheet, attached to the edge of the orbit, where it is continuous with the periosteum. In the upper eyelid, it blends with the superficial lamella of the aponeurosis of the levator palpebrae superioris, and in the lower eyelid with the anterior surface of the tarsus. It is perforated by the vessels and nerves which pass from the orbital cavity to the face and scalp.

Deep to the skin covering the anterior surface of the lid are bundles of striated muscle fibers of the orbicularis oculi muscle (see FIGS. 1 and 5). Some of the collagen fibers from the aponeurosis of the levator palpebrae muscle pass between these bundles to be inserted into the skin that covers the eyelid. Others connect with the tarsal plate, and still others continue toward the margin of the lid in front of the plate. This latter sheet of connective tissue becomes more areolar as it approaches the margin of the lid, which it reaches to form the gray line, a surgical landmark of some importance. Along this gray line, the lid may he split surgically, opening up the sub muscular space known to the ophthalmologist as the intermarginal space.

The hairs line the free margins of the eye lids are lined with the eye lashes. Eye lashes are attached in the free edges of the eyelids from the lateral angle of the eye to the lacrimal papillae. They are short, thick, curved hairs, arranged in 2, 3 or 4 rows in front of the gray line. Each upper eye lid contains 100-125 eyelashes though much fewer on the lower eyelid. The eye lashes are long and thick on the upper lid compared to lower lid lashes. The upper eye lashes curve upwards; those of the lower eyelid curve downwards and outwards so that the upper and lower eyelashes do not interlace when the lids are closed. The hair follicles of the eyelashes slant anteriorly as they pass to the surface. They are provided with sebaceous glands, named the glands of Zeis. Between the follicles, the sweat glands of Moll are disposed (FIGS. 1, 3-6). A sty is the result of the infection of either type of gland.

The palpebral fibers of the orbicularis oculi muscle are thin (FIGS. 1,5), pale in color and parallel with the palpebral fissure. Deep to the muscle there is a layer of loose areolar tissue (FIG. 1), which, in the case of the upper eyelid, is continuous with the subaponeurotic layer of the scalp, so that effusions of fluid (blood or pus) in this layer of the scalp can pass down into the upper eyelid. It is in this layer of the eyelids that the main nerves lie, so that local anesthetics have to be injected deep to the orbicularis oculi.

The tarsal glands are embedded in the thickness of the tarsi, and may be visible through the conjunctiva on everting the eyelids; they present an appearance like parallel strings of pearls (FIGS. 1, 3-6). They are yellow in color, arranged in a single row, and number about thirty in the upper eyelid, and rather fewer in the lower lids. They are embedded in grooves on the deep surfaces of the tarsi and correspond in length with the breadth of these plates; they are, consequently, longer in the upper than in the lower eyelid. Their ducts open on the free margins of the lids by minute foramina. The tarsal glands are modified sebaceous glands, each consisting of a straight tube with numerous small lateral diverticla (FIGS. 3-5). The tubes are supported by a basement membrane and are lined at their mouths by stratified epithelium; the deeper parts of the tubes and the lateral offshoots are lined by a layer of polyhedral cells. The secretion of the glands spreads over the margin of the eyelid and tends to prevent the tears from overflowing on to the cheek. It has also been suggested that it spreads over the external surface of the tear film and reduces evaporation.

Tears are produced by the lacrimal gland and several accessory tear glands at the upper part of the upper eye lids (a total of 57 in each lid, FIG. 3). The main lacrimal gland lies in the superolateral corner of the bony orbit. Less than a dozen ducts run from the gland to empty along the superior formix. Small accessory tear glands, the glands of Krause, are scattered along both formices. Still smaller glands are present in the caruncle (see FIGS. 1, 3 and 4). It is of interest that the eye may remain healthy in the absence of the lacrimal gland; this suggests that the function of the gland is to some extent that of providing floods of tears on special occasions and other accessory glands can provide the same lubrication and wetting as lacrimal glands. The secretion of the tear glands is slightly alkaline. In addition to various salts, it contains an enzyme, lysozyme, which is bactericidal. Tears spread evenly over the cornea and the conjunctiva by the blinking of the lids, which keep the surface of the cornea and the conjunctiva moist. Floods of tears assist in washing foreign particles from the conjunctival sacs and the cornea.

Diseases Causing Loss of Eye Lashes and Eye Brows

Eyelash and eyebrow loss may not be the most common form of hair loss, but it can be a stressful problem in those who are appearance and beauty-conscious. Eyelashes are considered a sign of beauty in all cultures, enough so that cosmetic companies have entire lines of eyelash enhancing products and have proven to be a big business. Cleopatra, a historic ruler of ancient Egypt contributed more to the consciousness of woman to notice eye lashes as part of their beauty and cosmetic make up. Any acute, chronic systemic and local eye lid disease can result in loss of eye lashes (Ciliary Madarosis). The biggest culprit is the treatment of cancer with use of massive doses of chemotherapy which affects inhibits or kills the multiplying cells of the hair bulb and follicle. The following conditions can result in eyelash and eyebrow thinning and/or loss:

Aging can easily cause eyelash loss as it happens to the hair on the head.

Alopecia greata is a medical condition where body's immune system starts attacking hair follicles which will affect hair on the entire body, including eyelashes.

Blepharitis infection attack of lids repeatedly can cause eyelash loss. It causes swelling and itching on the eyelid due to excessive bacteria growth in tiny oil glands. This will not cause general hair loss, but will cause loss of eyelashes, especially if it goes untreated. Ciliary Madarosis is the medical term for eyelash loss, which is caused by blepharitis and should be cared for by your physician with antibiotics and bactericidal agents.

Allergic reaction to the various skin and eye makeup products.

Demodex folliculorum is a type of mite than exists naturally in skin pores and hair follicles. These mites cause eyelashes to fall out.

General infection caused by a variety of reasons can swell the eyelid, sometimes forcing the eyelash to fall out. Septic shock and extreme stress can result in eyelash loss.

Hyper or Hypothyroidism can mean an overactive or underactive thyroid can cause areas of hair to thin or fall out, including eyelashes.

Improper care such as using harsh cosmetics or forgetting to remove eye makeup before bed can cause eyelashes damage. They can become brittle and thus break or fall out.

Poor diet is always a cause for general hair thinning or hair loss such as serious bulimia, which wrecks the body hair including scalp and eye lash hair.

Trauma to the eyelid can result is loss of eyelashes including ripping the eyelashes out to something as serious as burning off the eyelashes. Usually, though, eyelash trauma means the eye was being repeatedly rubbed and scratched until the eyelash was inadvertently pulled loose.

Medications are a frequent cause of eyelash loss including some heart and blood pressure medications, thyroid medications, cholesterol lowering drugs etc. Chemotherapy for treatment of cancer is the worst offender of eyelash loss. The following drugs, medications, substances or toxins may possibly cause eye lashes loss such as Acitretin (Soriatane, Neotigason), chemotherapeutic agents, corticosteroids, estrogens etc.

Other causes of eyelash growth and loss: Ichthyosiform erythroderma (nonbullous congenital, Ichthyosis), Keratosis Follicularis Spinulosa Decalvans, Leprosy, Progeria, Siemens syndrome, Spastic paraplegia—neuropathy—poikiloderma, anorexia nervosa, chronic anorexics, mental patients, diseases affection general nutritional intake etc.

### SUMMARY OF THE INVENTION

A method of enhancing eyelash and eye brow hair growth utilizes insulin and/or IFG-1 alone or in combination with other known hair growth promoting therapeutic, pharmaceutical, biochemical and biological agents to increase the effect of various therapeutic hair regrowth strategies to enhance eyelash and eye brow hair growth.

Other features and advantages of the instant invention will become apparent from the following description of the invention which refers to the accompanying drawings.

### BRIEF DESCRIPTION OF THE DRAWINGS

FIG. 1 is a diagram of a low resolution microscope cross section view of the upper eyelids showing the eyelid and its histological components which make eyelid structures.

FIG. 2 shows the detailed histology of the hair follicle, hair bulb and hair shaft and their contribution to hair growth along with stem cells, germinal cells of the matrix which contributes to the healthy hair growth.

FIG. 3 is a diagrammatic presentation of the eyelid with various glands, modified terminal hairs on the outer surface and vellus hair at the edge of the upper eyelid located in front of the opening of the Meibomian glands.

FIG. 4 shows the site of application of the hair growth therapeutic agents described in this invention.

FIG. 5 shows the site of application of the hair growth therapeutic agents described in this invention.

FIG. 6 shows the site of application of the hair growth therapeutic agents described in this invention.

FIG. 7 shows the effect of application of therapeutic agents described in this invention and the rate of hair growth from the start in a timely fashion.

FIG. 8 shows the effect of application of therapeutic agents described in this invention and the rate of hair growth from the start.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

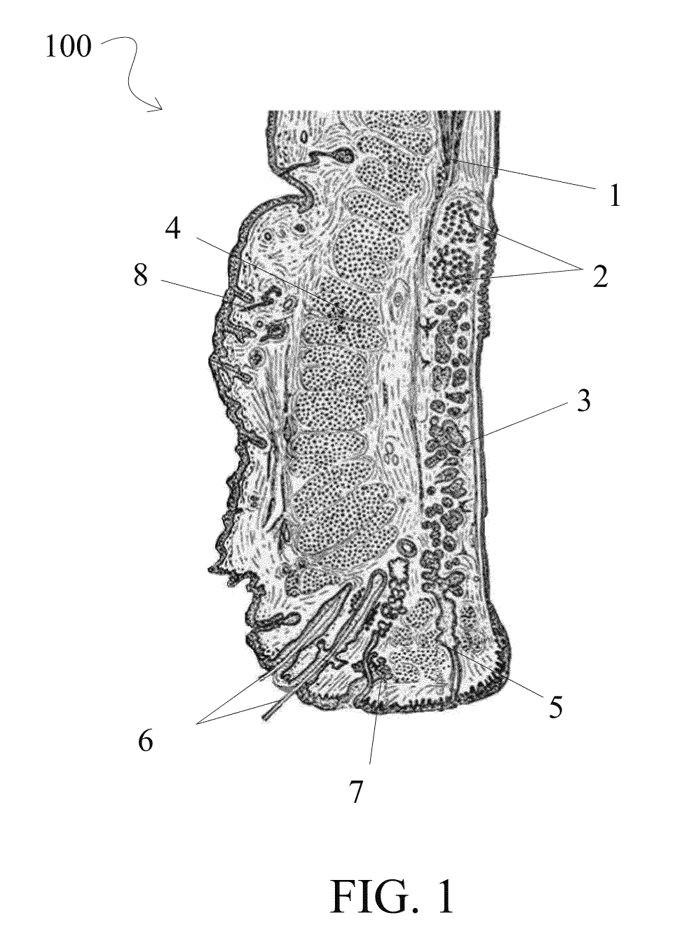

Referring to FIG. 1, a drawing of a low resolution microscope cross section view of the upper eyelids 100 shows levator aponeurosis (1) enters between the orbicularis oculi muscle and the conjunctival surface. Accessory lachrymal glands of Wolfring are shown (2). The meibomian glands (3) of the tarsal plate produce the lipid that will line the layer of the tear film. The Meibomian lids empty into ducts that dot the marginal surface of the eyelid (5) and can be seen emanating droplets of oil for the tears. The orbicularis muscle (4) is striated muscle that is responsible for blinking and squeezing eyelids shut. The cilia or eyelashes (6) emanate from the lid immediately adjacent to apocrine glands of Moll (7). The skin surface (8) of the eyelid is the thinned epidermis in the body and contains vellus hair and adnexa (from Grey's anatomy).

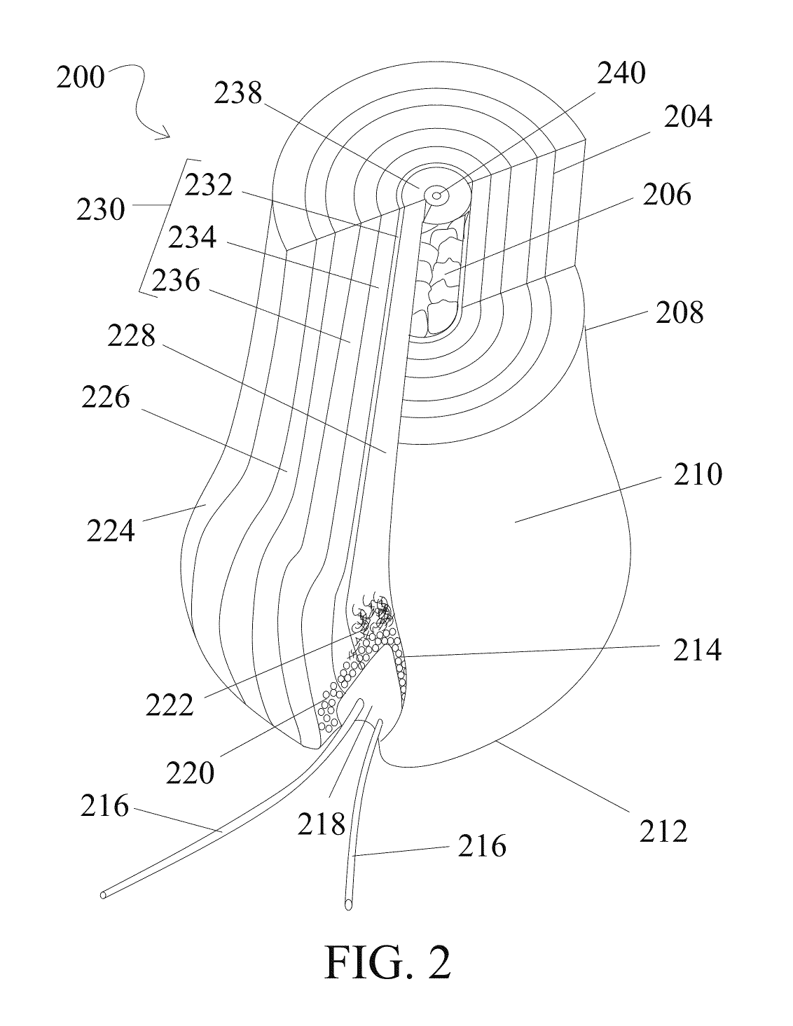

FIG. 2 is a diagrammatic presentation of the structure of the hair follicle 200, hair bulb 210 embedded in the follicle 212, hair root 208, and hair shaft. Note the location of the stem cells, matrix germinal cells which contribute to the hair growth of the eye lashes, and the location of melanocytes responsible for hair color. Also shown are glossy membrane 204, germinal cells 214, papillar blood vessel 216, dermal papilla 218, dividing stem cells 220, melanocytes 222, dermis 224 connective tissue, external root sheath 226, zone of keratinization 228, inner root sheath 230, cuticle of inner root sheath 232, Huxley's layer 234, Henie's layer 236, 238 cortex and medulla 240.

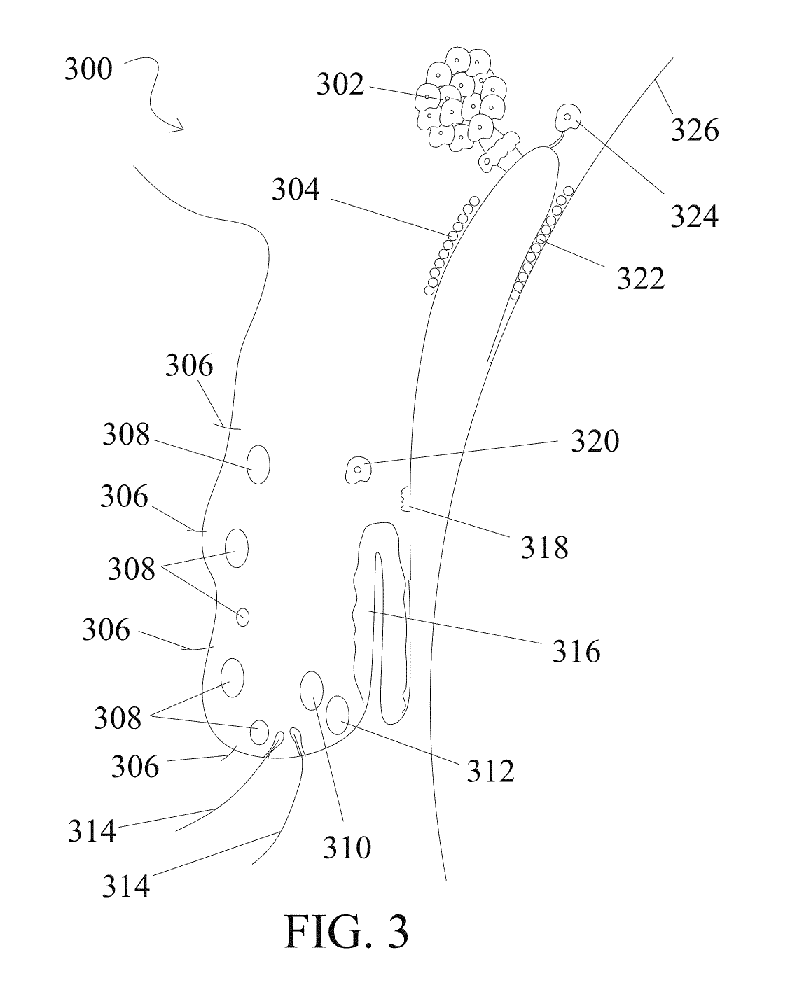

Now referring to FIG. 3, a diagrammatic presentation of the eye lid with various glands 300 in the upper eyelids, modified terminal hairs on the outer surface and vellus hair 306 at the edge of the eye located in front of the opening of the Meibomian glands 316. Also shown are the lacrimal glands 302, goblet cells 304, sweat gland 308, glands of Moll 310, glands of Zeis 312, eyelash 314, crypts of Henle 318, lacrimal glands of Wolfring 320, bulbar conjunctiva 322, lacrimal glands of Krause 324 and cornea 326.

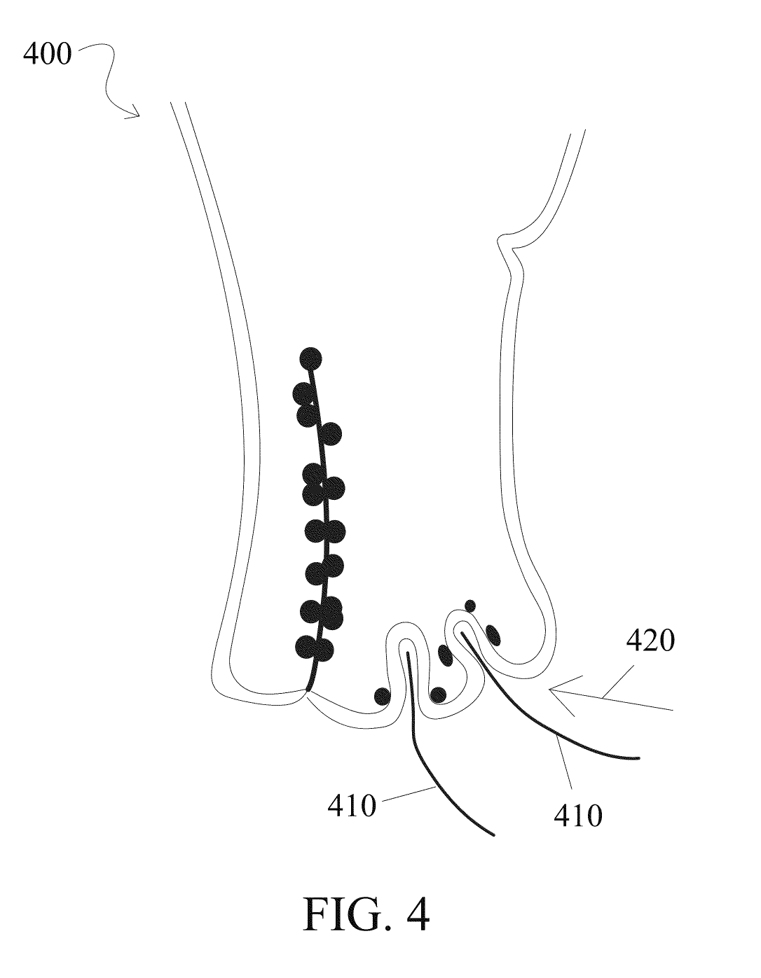

Referring now to FIG. 4, a diagrammatic presentation of the eyelid margins with rows of eye lashes 400 is presented to illustrate an application site. Arrow 420 points to the site of therapeutic agents' application for eyelash hair growth.

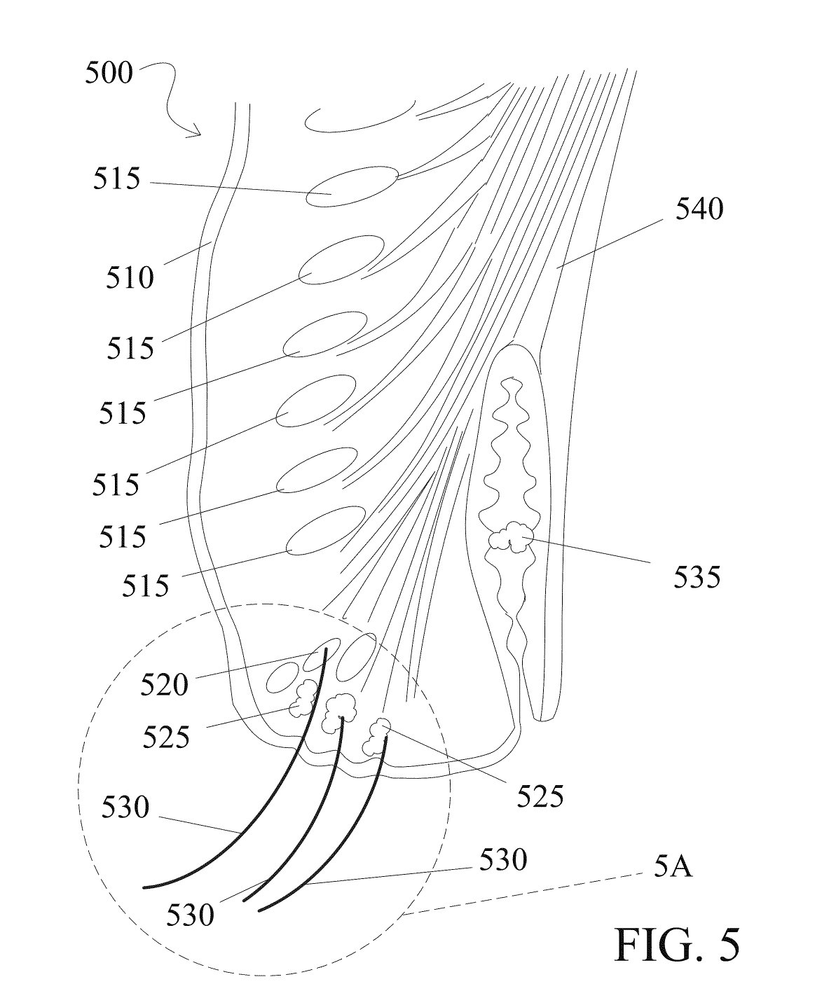

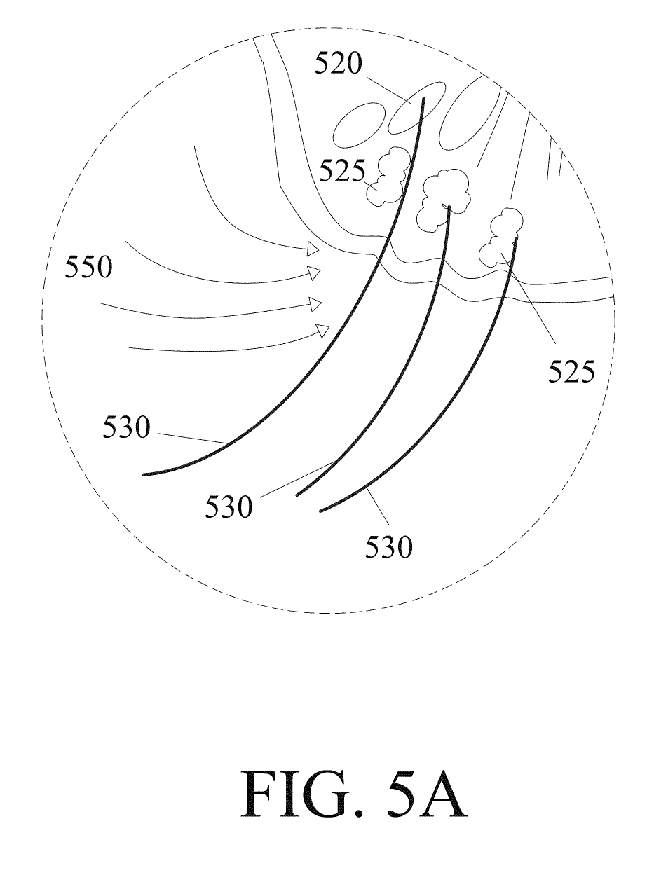

Referring now to FIGS. 5 and 5A, a diagram of the eyelid margins with rows of eye lashes 500 and the site of therapeutic agents' application (arrows) for eye lash hair growth is shown with skin 510, orbicularis oculi muscle 515, hair follicle 520, sebaceous gland 525, eyelash 530, Meibomian gland 535, tarsal plate 540 and application site 550.

Scalp, eyebrows and eyelashes hair loss or Alopecia (baldness) is a shortage or paucity of normal terminal hair. It is a deficiency of terminal hair or modified terminal hair, their broad diameter (thickness) and the colored hair that is readily seen in this condition. Although there is a noticeable absence of terminal hair, the eyelid skin does contain vellus hair which is fine colorless hair which may require magnifier to find them in the scalp and the eyelids. The vellus hair in the balding scalp and the eyelids has the capacity to rejuvenate with proper therapy as described in this invention. Along with balding scalp; due to any number of above described reasons, the eyelids may lose their hair lashes. This condition can be very distressing to cosmetically conscious females. It is one thing to have a hair growth stimulator, but it is another part to get it to the site of hair growth, i.e. hair follicle and hair bulb. We describe such agents in this invention and their site of application.

Besides aspirin and antibiotics, insulin is the most commonly used therapeutic agent known to the public and professional people. It has been used in home by the patients or in the office by the physicians. It can be easily obtained and used for hair growth as described in this invention. Insulin is a hormone secreted by beta cells in the islets of Langerhans in the pancreas. It activates and participates in all the metabolic pathways in the normal and disease afflicted cells; however, it can lead to increased DNA, RNA and protein synthesis which result in increased growth by mitosis (Osborne C K, et al. Hormone responsive human breast cancer in long-term tissue culture: effect of insulin. Proc Natl Acad Sci USA 1976; 73: 4536-4540); enhances the permeability of cell membranes to many therapeutic agents (hair growth therapeutic agents) besides glucose, and electrolytes.

It helps and facilitates to move the therapeutic, pharmaceutical, biochemical and biological agent molecules from extra cellular fluid (ECF) to intracellular fluid (ICE) meaning from outside the cells to inside the cells. Insulin is anabolic trophic factor needed for the growth, multiplication, of all cells in the body including hair growth stem cells in the hair follicle and multiplying matrix hair bulb cells of the eye lashes. Increased cellular metabolic activity induced by insulin also enhances the uptake and augments the action of all therapeutic agents, biological and pharmacological agents by the cells and inside the cell including the cells responsible for eye lashes growth.

Insulin enhances the concentration and therapeutic effectiveness of pharmaceutical, biochemical and biological agents which has disease curing besides hair growing effect. Once inside the cells; the insulin enhances, and expands the effectiveness of any and all therapeutic agents including that agent proven to promote the hair growth such as minoxidil, finasteride and Latisse—a prostaglandin preparation.

Insulin itself enhances the hair growth on its own right. This is further supported by the observation of hair loss in diabetics and hair growth with hyperinsulinism. Our body has certain metabolic cycles, and one of these is the hair growth cycle as explained above. An individual hair will grow for several years and then enter a resting phase before being shed. In a healthy non-diabetic person, this fallen hair regrows quickly. However, diabetes affects the normal metabolic cycles of our body, including the hair growth cycle.

If this cycle is disrupted, hair that is shed normally may not regrow right away, or it may not regrow at all. Diabetes can also affect hair growth in these ways: This disease weakens immune system, increasing the susceptibility to infections, including scalp and eye lids infections, which can cause hair loss. Diabetes also adversely affects the circulatory system (ASVD), causing problems all over your body, including hair follicles of the scalp, eye ashes and eyebrows. Hair follicles that don't get enough nutrients and oxygen because of circulatory problems can't grow, produce new hairs, and the follicles may even die from lack of nutrition. Interestingly, the high levels insulin the blood in some diseases and diabetics can result in hypertrichosis.

In our decade of studies and medical practice; we found, there is not a single disease which cannot be treated using Insulin to enhance the therapeutic effectiveness of the pharmaceutical, biochemical and biological agents and compounds including hair loss and augmentation of hair growth. In an ingenious vitro studies, it has been conclusively and methodically demonstrated that the Insulin activates and modifies metabolic pathways in MCF-7 human breast cancer cells, and increase the cytotoxic effect of methotrexate up to 10,000 fold (Oliver Alabaster' et al. Metabolic Modification by Insulin Enhances Methotrexate Cytotoxicity in MCF-7 Human Breast Cancer Cells, Eur J Cancer Clinic; 1981, Vol 17, pp 1223-1228. Richard L. Schilsky and Frederick. S. Ordway. Insulin effects on methotrexate polyglutamate synthesis and enzyme binding in cultured human breast cancer cells. Cancer Chemotherapy Pharmacol (1985) 15: 272-277).

Research studies in human breast cancer and my own studies on every kind of cancer and infection in any part of the body have shown that the group treated with insulin plus low dose methotrexate and other anticancer agents (and or antibiotics for infection) responded better than the patient treated with insulin or chemotherapy alone. (Eduardo Lasalvia-Prisco et al. Insulin-induced enhancement of antitumoral response to methotrexate in breast cancer patients. Cancer Chemotherapy Pharmacol (2004) 53: 220-224. Ayre S G, Perez Garcia y Bellon D, Perez Garcia D Jr (1990) Neoadjuvant low-dose chemotherapy with Insulin in breast carcinomas. Eur J Cancer 26:1262-1263; T. R. Shantha presented at Cancun IPT meeting 2nd meeting 2004 and unpublished studies).

These observations supports the hypothesis that disease or healthy cell sensitivity to therapeutic, pharmaceutical, biochemical and biological agents such as those to be used to enhance the hair growth could be increased by using the method described in this invention using insulin, IGF-I. Our study of injecting Insulin followed by anticancer chemotherapeutic agents directly into cancer masses on hundreds of advanced and localized cancers supports this finding. Using this method, the palpable tumors including enlarged lymph nodes literally melted away. We also used plain insulin locally as a therapeutic agent in chronic wounds, periodontal disease, post surgical wound healing, many eye and ear diseases etc which will be reported later.

The hormone IGF-1 (insulin-like growth factor 1) is a growth hormone that is believed to work with androgens to initiate and maintain hair growth. Working with androgens, IGF-1 may induce hair growth factors (Itami S, Kurata S, Takayasu S. Androgen induction of follicular epithelial cell growth is mediated via insulin-like growth factor-I from dermal papilla cells. Biochem Biophys Res Commun 1995 Jul. 26; 212(3):988-94). Researchers also suspect that IGF-1 may be able to stimulate the growth hair follicle cells directly (Su H Y, Hickford J G, Bickerstaffe R, Palmer B R. Insulin-like growth factor 1 and hair growth. Dermatol Online J 1999 November; 5(2):1), and may regulate hair growth and the hair growth cycle on its own (Hembree J R, Harmon C S, Nevins T D, Eckert R L. Regulation of human dermal papilla cell production of insulin-like growth factor binding protein-3 by retinoic acid, glucocorticoids, and insulin-like growth factor-1. J Cell Physiol 1996 June; 167(3):556-619). So the role of IGF-1 (also known as Somatomedin-C) in hair health cannot be ignored and can be used to enhance the growth of eye lashes as described in this invention.

Insulin-like growth factor I (IGF-I) accelerates, in a concentration-dependent manner, growth of hair and hair follicles. Insulin-like growth factor (IGF)-I and -II had no significant effect on hair follicle growth when maintained in the presence of 10 micrograms/ml insulin. However, in the absence of insulin, both IGF-I (0.01-100 ng/ml) and IGF-II (0.01-100 ng/ml) stimulated hair follicle growth in a dose-dependent manner. IGF-I was more potent than either insulin or IGF-II, stimulating maximum rates of hair follicle growth at 10 ng/ml, whereas IGF-II gave maximum stimulation at 100 ng/ml. The rates of hair follicle growth stimulated by 10 ng/ml IGF-I were identical to those stimulated by 10 micrograms/ml insulin. IGF-II (100 ng/ml), however, was unable to stimulate hair follicle growth to the same extent as insulin. Both IGF-I (10 ng/ml) and IGF-II (100 ng/ml) were more potent than insulin at preventing hair follicles from entering into a catagen-like state. Relaxin is a member of the insulin family of polypeptide hormones and exerts its best understood actions in the mammalian reproductive system and may have an effect on rapid hair growth when combined with insulin and other known therapeutic, pharmaceutical, biochemical and biological agents.

Growth hormone had no effect on hair follicle growth or morphology in the absence of insulin. These data suggest that in vitro IGF-I may be an important physiologic regulator of hair growth and possibly the hair growth cycle.

The actions of IGF-I are modulated by proteins produced in dermal papilla cells which bind IGF (insulin-like growth factor-binding proteins: IGFBPs) which acts as mitogen. The exact mechanism of modulation has not yet been resolved (Batch J A, Mercuri F A, Werther G A. Identification and localization of insulin-like growth factor-binding protein (IGFBP) messenger RNAs in human hair follicle dermal papilla. J Invest Dermatol 1996; 106: 471-5).

However, it has been shown that IGFBP-3 (which is the most abundant IGFBP type in dermal papilla cells) forms a complex with free IGF-I to reduce the concentration of IGF-I available for stimulation of hair elongation and maintenance of the anagen phase (Hembree J R, Harmon C S, Nevins T D, et al. Regulation of human dermal papilla cell production of insulin-like growth factor binding protein-3 by retinoic acid, glucocorticoids, and insulin-like growth factor-1. J Cell Physiol 1996; 167: 556-61). Retinoids and glucocorticoids stimulate production of IGFBP-3 in dermal papilla cells reducing the hair growth.

Insulin stimulated hair follicle growth in a dose-dependent manner over the range of 0.01 to 100 micrograms/ml. Maximum rates of hair follicle growth were observed when follicles were maintained in medium containing 10 micrograms/ml insulin, which is supraphysiologic. Hair follicles maintained in the absence of insulin or at physiologic levels showed premature entry into a catagen-like state (Philpott M P, Sanders D A, Kealey T. Effects of insulin and insulin-like growth factors on cultured human hair follicles: IGF-I at physiologic. J Invest Dermatol 1994; 102: 857-61). Insulin itself has the same effect as IGF-I. It has been observed that body hair in patients with hyperinsulinism has a male distribution pattern (Fossati P, Fontaine P. Endocrine and metabolic consequences of massive obesity. Rev Prat 1993; 43: 1935-9. Hrnciar J, Hrnciarova M, Jakubikova K, et al. Insulin resistance and arterial hypertension. Hyperinsulinism as a basic etiopathogenic factor in essential arterial hypertension and associated phenomena. Vnitr Lek 1992; 38; 868-78).

Hyperinsulinism as a basic etiopathogenic factor in essential arterial hypertension and associated phenomena. Vnitr Lek 1992; 38: 868-78). Insulin has profound effect on cell mitosis and metabolism as described above. How effective is the insulin in hair growth has been demonstrated that the hair follicle is an aerobic glycolytic, glutaminolytic tissue needing insulin for its health and growth. (Philpott M P, Sanders D, Westgate G E, Kealey T. Human hair growth in vitro: a model for the study of hair follicle biology. J Dermatol Sci. 1994 Jul. 7 Suppl:S55-72).

In an important experiment, Zheng et al showed the role of insulin like growth factor-I which have insulin like effect; induced the inner ear epithelial cell culture growth (Zheng, J. L., Helbig, C. & Gao, W-Q. Induction of cell proliferation by fibroblast and insulin-like growth factors in pure rat inner ear epithelial cell cultures. J. Neurosci. 17:216-226 (1997). There is a clear indication that insulin and IGF-I not only play a role in potentiation of pharmacological therapeutic effect of pharmaceutical, biochemical and biological agents, they can independently stimulate cells (inner ear hair cells) growth besides stimulating metaplasic-dysplasic cells (T. R. Shantha, Unknown Health Risks of Inhaled Insulin. Life Extension, September 2007 pages 74-79, Post publication comments in September 2008 issue of Life Extension, Pages 24. T. R. Shantha and Jessica G. Inhalation Insulin, Oral and Nasal Insulin Sprays for Diabetics: Panacea or Evolving Future Health Disaster. Part I: Townsend Letter Journal: Issue #305, December 2008 pages: 94-98; Part II: Townsend Letter, January, 2009, Issue # 306, pages-106-110).

On the other hand; growth hormone (somatotropin) alone has no direct influence on hair follicle and hair growth. It needs insulin to have exert its trophic effect (Philpott M P, Sanders D A, Kealey T. Effects of insulin and insulin-like growth factors on cultured human hair follicles: IGF-I at physiologic. J Invest Dermatol 1994; 102: 857-61). Greying or whitening of hair is due to the collection of minute air bubbles in the cortex and medulla of the hair shaft and due to loss of pigment (melanin) formation by cells in the germinal matrix.

It is a known physiological phenomenon that the insulin does bind to the receptor sites of the IGF-I and insulin receptors and exert multiple profound physiological effects (induce cell growth besides glucose transport) such as enhanced metabolism, enhances mitosis, enhances the therapeutic effect of other pharmacological agents as reported (T. R. Shantha, Life extension September 2007 article) on the cell to which it binds has been reported in above publications. This physiological effect of insulin is used to turn the non dividing resting cancer cells to divide and make them sensitive to radiation therapy as well as anti metabolic chemotherapy agents and antibiotics. The same principle is used in the treatment of hair loss as described below in our invention.

Insulin, potassium and glucose are routinely administered to treat low potassium levels in the cells even to this day. Insulin and glucose facilitates the entry of potassium inside the cell—a life saving measure. We have used insulin as potentiator of uptake and enhancer of therapeutic action of diverse therapeutic agents to cure and/or curtail curable acute, chronic and incurable diseases such as cancers, Lyme disease, scleroderma, antibiotic resistant staphylococcus infection, MRSA infection, chronic wounds, neurological diseases, inner and middle ear affliction, autoimmune diseases and many other diseases with good results.

We have used insulin for more than a decade to enhance the effectiveness of locally injected therapeutic agents specially cancers with chemotherapeutic agents with remarkable results. Our unpublished data also supports that the insulin spray on indolent ulcers anywhere in the body, including the oral (gums) and nasal cavity. It stimulated the fibroblast, endothelial cell and skin cell growth resulting accelerated wound healing. Application of insulin soaked cotton swabs (1-3 units in normal saline) after teeth extraction induces rapid healing with reduced pain. Our unpublished studies show that the application of insulin and antibiotics such as doxycycline locally on the gums eliminated gum diseases (periodontitis) which is under study.

Insulin is a metabolic activity enhancer of all cells and therapeutic agents. Hence it can play an important role in treatment of hair loss (Dr. T. R. Shantha; 1. discovery of insulin and IPT: amazing history, 2. high dose methotrexate therapy using Insulin; 3. local injections of tumors with insulin and cytotoxic drugs; 4. two and three cycle insulin Potentiation therapy: Presented at 2^{nd} international conference on Insulin Potentiation Therapy held at Cancun, Mexico, Jun. 28-Jul. 1, 2004). A synergy between certain membrane and metabolic effects of insulin on cell molecular biology increases therapeutic efficacy, and it does so with reduced doses of the drugs, enhancing their uptake with augmented greater than before therapeutic efficacy and increasing safety. This treatment strategy has been applied abroad over the last 7 decades with very promising clinical results (1939 U.S. Pat. No. 2,145,869 by Dr. Donato Perez Garcia).

It is known that the pharmaceutically acceptable oxidizing agent facilitates the delivery of the bioactive agent through the skin and mucous membranes. In general, the oxidizing agent can react with molecules present in the skin that would adversely react with the bioactive agent. For example, reduced glutathione present in the skin and mucus membranes of the eye lids can inactivate bioactive agents such as insulin by breaking chemical molecular bonds. Not wishing to be bound by theory, when delivering insulin transdermally, reduced glutathione can inactivate insulin.

Specifically, insulin has numerous disulfide bonds which are crucial for its protein conformation, biological activity, and subsequent therapeutic effects. Reduced glutathione will inactivate insulin by reducing or breaking insulin's disulfide bonds. Once these disulfide bonds are broken; insulin becomes inactive due to lost protein conformation and biological activity. Thus, the administration of the oxidant or oxidizing agents by spray, ointment, cream, or solution application or patch using the devices (as described by Shantha et al in U.S. PATENT APPLICATION PUB. NO.: 2009/0347776 A1) herein prevents the inactivation of the bioactive agent such as insulin when applied to the skin and eyelid edges. Specifically, applying an oxidant or a pharmaceutically oxidizing agent transdermally will lower or prevent the effects reduced proteins and reduced biological molecules have on the bioactive agents.

In this manner, the inactivation of bioactive agents via reduction or cleavage of crucial molecular bonds will be avoided. The selection and amount of the pharmaceutically acceptable oxidizing agent can vary depending upon the bioactive agent that is to be administered. In one aspect, the oxidizing agent includes, but is not limited to, iodine, povidone-iodine, any source of iodine or combinations of oxidants, silver protein, active oxygen, potassium permanganate, hydrogen peroxide, sulfonamides, dimethyl sulfoxide (DMSO) or any combination thereof. These oxidizing agents may also act as absorption agents which help facilitate delivery of a therapeutic agent onto and into a skin.

In one aspect, the oxidant is at least greater than 1% weight per volume, weight per weight, or mole percent. In another aspect, the mucosal membrane permeability enhancer may be at least greater than 1.5%. 2.0%. 20 2.5%, 3.0%, 3,570, 4.0%, or 4.5% weight per volume, weight per weight, or mole percent. In this aspect, the oxidant may range from 2% to 10%, 2% to 9.5%, 3% to 8%, 3% to 7% or 4% to 6% weight per volume, weight per weight, or by mole percent. Additional components can be present in the device to facilitate the delivery of the bioactive agent transdermally to the subject.

In one aspect, transdermal penetration enhances can be used to further expedite the entry of the bioactive agent into the hair follicle including hair papilla which is responsible for hair growth. Penetration enhancers not only penetrate a membrane efficiently, but these enhancers also enable other bioactive agents to cross a particular membrane more efficiently. Penetration enhancers produce their effect by various modalities such as disrupting the cellular layers of the alopecia skin surface interacting with intracellular proteins and lipids, or improving partitioning of bioactive agents as they come into contact with the mucosal membranes.

With these enhancers, macromolecules up to 10 kDa are able to pass through the epidermal layers of skin to dermal layer reaching the hair papilla, follicle and bulb. These enhancers should be non-toxic, pharmacologically inert, non-allergic substances. In general these enhancers may include anionic surfactants, ureas, fatty acids, fatty alcohols, terpenes, cationic surfactants, nonionic surfactants, zwitterionic surfactants, polyols, amides, lactam, acetone, alcohols, and sugars. In one aspect, the 10 penetration enhancer includes dialkyl sulfoxides such as dimethyl sulfoxide (DMSO), decyl methyl sulfoxide, dodecyl dimethyl phosphine oxide, octyl methyl sulfoxide, nonyl methyl sulfoxide, undecyl methyl sulfoxide, sodium dodecyl sulfate and phenyl piperazine, or any combination thereof. In another aspect, the penetration enhancer may include lauryl alcohol, diisopropyl sebacate, oleyl alcohol, diethyl sebacate. dioctyl sebacate, dioctyl azelate, hexyl laurate, ethyl caprate, butyl stearate, dibutyl sebacate, dioctyl adipate, propylene glycol dipelargonate, ethyl laurate, butyl laurate, ethyl myristate, butyl myristate, isopropyl palmitate, isopropyl isostearate, 2-ethylhexyl pelargonate, butyl benzoate, benzyl benzoate, benzyl salicylate, dibutyl phthalate, or any combination thereof. In one aspect, the skin permeability enhancer is at least greater than 1% weight per volume, weight per weight, or mole percent. In another aspect, the mucosal membrane permeability enhancer may be at least greater than 1.5%, 2.0%, 2.S %, 3.0%. 3.5%. 4.0%. 4.5% up to 50% weight per volume, weight per weight, or mole percent. In one aspect, the mucosal membrane permeability enhancer is dimethyl sulfoxide. In this aspect, the amount of dimethyl sulfoxide may range from 2% to 10%. 2% to 9.5%. 3% to 8%. 3% to 7%, or 4% to 6% weight per volume, weight per weight, by mole percent, or any effective therapeutic amount.

In other aspects, these additional components may include antiseptics, antibiotics, anti-vitals, anti-fungals, anti-inflammatories, anti-parasitic anti-dolorosa, antihistamines, steroids, vasodilators and/or vasoconstrictors within the device to reduce inflammation, irritation, or reduce rapid absorption through the alopecia skin to the root of the hair which need to be activated to stimulate the hair growth and restore the hair loss. Such vasoconstrictors may include phenylephrine, ephedrine sulfate, epinephrine, naphazoline, neosynephrine, vasoxyl, oxyrnetazoline, or any 5 combination thereof. Such anti-inflammatories may include non-steroidal anti-inflammatory drugs (NSAIDs). NSAIDs alleviate pain and inflammation by counteracting cyclwxygenase and preventing the synthesis of prostaglandins.

In one aspect. NSAIDs include celecoxib, meloxicam, nabumetone, piroxicam, napmxen, oxaprozin, rofecoxib, sulindac, ketoprofen, valdewxid, anti-tumor necrosis factors, 10 anti-cpokines. anti-inflammatory pain causing bradykinins or any combination, thereof. Such antiseptics, anti-vitals, anti-fungals, and antibiotics, may include ethanol, propanol, isopropanol, or any combination thereof; a quaternary ammonium compounds including, but not limited to, benzalkonium chloride, cetyltrimethylammonium bromide, cetylpyridinium chloride, benzethonium chloride, or any combination thereof: boric acid; chlorhexidine gluconate, hydrogen peroxide, iodine, mercurochrome, ocetnidine dihydrochloride, sodium chloride, sodium hypochlorite, silver nitrate, colloidal silver, mupirocin. erthromycin, clindamycin, gentamicin, polymyxin, bacitracin, silver, sulfadiazine, or any combination thereof.

Alopecia is also contributed by the hair follicle, scalp, eye brows, and eye lash infection. It is intent of this invention to use of the insulin along with above described anti-inflammatory antibacterial agents that can eliminate one of the causes of hair loss and restore normal eyelash hair growth.

In accordance with one aspect of the invention, the compound used to apply locally to the eye lids site are mixed with a dermatologically well-suited vehicle or carrier. The compositions of this invention may comprise aqueous solutions such as e.g., physiological saline, oil, gels, patches, solutions or ointments. The vehicle which carry these biologically active therapeutic agents may contain dermatologically compatible preservatives such as e.g., benzalkonium chloride, surfactants like e.g., polysorbate 80, liposomes or polymers, for example, methyl cellulose, polyvinyl alcohol, polyvinyl pyrrolidone and hyaluronic acid etc.

There are various forms of insulin used to treat diabetes. They are grouped under rapid, short, intermediate, and long acting insulin as shown below in Table 1. It is also dispensed as premixed form containing rapid to long acting insulin. We use rapid and short acting insulin as noted in the following table to enhance the eye lashes hair growth.

TABLE 1

| Type of Insulin & | Onset | | Duration of |
|---|---|---|---|
| Brand or trade Names | of action | Peak effect | action |
| Rapid-Acting or ultra short | | | |
| acting Insulin | | | |
| Humalog or lispro | 15-30 min. | 30-90 min | 3-5 hours |
| Novolog or aspart | 10-20 min. | 40-50 min. | 3-5 hours |
| Apidra or glulisine | 20-30 min. | 30-90 min. | 1-2½ hours |
| Short-Acting Insulin | | | |
| Regular (R) humulin or | 30-60 min. | 2-5 hours | 5-8 hours |
| novolin | | | |

We found the use of rapid acting insulin marketed as Humalog or Novolog, regular humulin or novolin are best suited for treatment of eye lash growth. Our studies also found that local application of rapid action or other insulin formulations did not change the blood sugar levels indicating it is safe to apply 3-5 IU insulin to the eye lids.

Hair Growth Stimulant and Examples of How to Use Our Invention

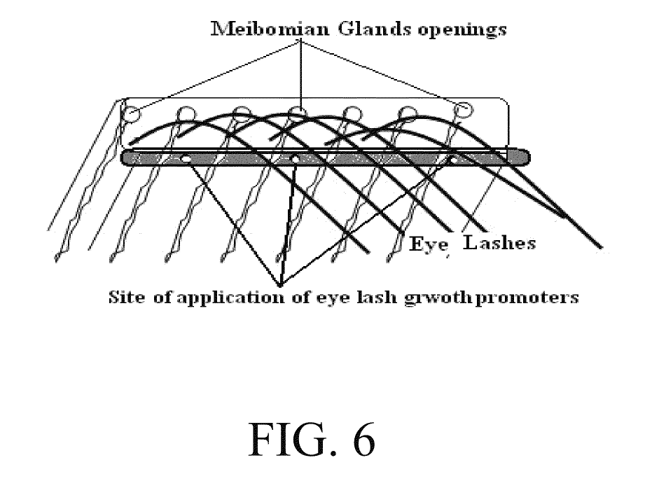

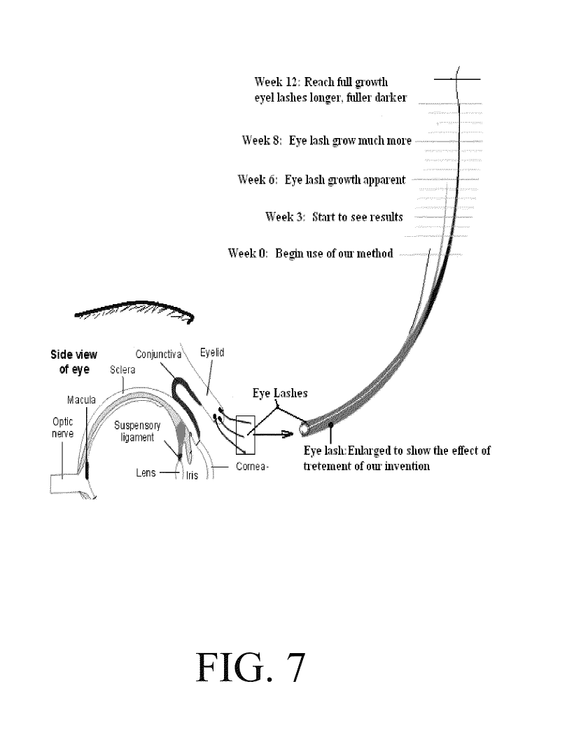

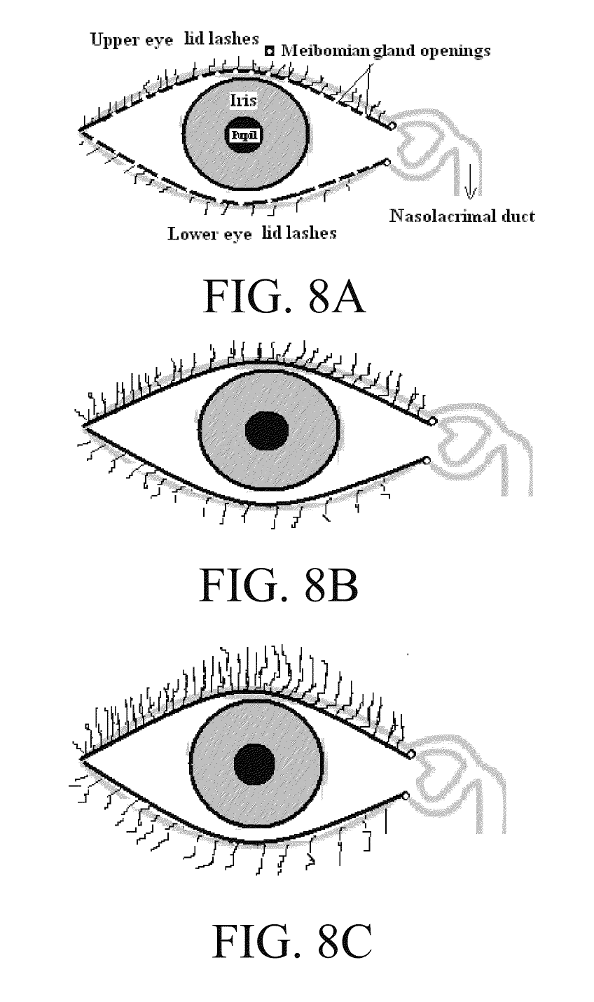

The therapeutic agents described in this invention are applied at the emergence of eye lashes at the edge of the eye lids as shown in the FIGS. 1, 3-8. It is important to avoid the therapeutic compounds coming in contact with the eye ball itself. If they do, washing the eye ball with clean cold water is in order to remove them coming in contact with delicate cornea and the rest of the eye ball.

Wash the face and eye surface (eyelids) with mild soap. This is followed by application of oxidant. In our invention, we can use any one of the above described antioxidants, but in this instance we are discussing povidine iodine. We have also used insulin application at the emergence of the eyelashes without the use of oxidants which is as effective. Allow it to dry, and follow it by application of insulin at the root of the eye lashes only repeatedly 2 to 3 times as it dries.

This can be followed by application of hair growth therapeutic agents as described below which are already available FDA approved and/or those about to be approved or not approved. This process can be repeated every 12 or 24 hours for 3-7 days a week till the desirable results are obtained which may take up to 12 weeks. Once the objective is achieved, use the method 2 to 3 times weekly before going to bed to keep the eye lashes hair growth stimulation.

The hair growth stimulant of the present invention is obtained which are already in the market or under experiment. Eyelash growth therapeutic agents are used under the following conditions:

1. To grow the eyelashes back which were shed due to disease and other health conditions affecting the eyelashes hair growth. Before starting the eyelashes enhancing therapy, one needs to find out the underlying cause for madarosis (the loss of eyelashes). Once the underling etiology is found, treat the disease and then use the eyelash growth product described in this invention.

2. The second intent of the use eyelash hair growth product is purely cosmetic and they need guidance how to use it.

Preparations before Application of Eye Lash Hair Growth Products of this Invention

Eyelash growth therapeutic agents should be used once a day or at the most no more than three times a day. They should be applied preferably at night time before going to bed. First the person must remove all eye make-up and contact lenses. Make sure eyelash and the eyelid skin is clean, dry, and free of any oils used to remove the make-up. Put a drop of the solution on one of the applicators or dip the applicator in the solution supplied. Then use the sterile applicator to apply the solution at the base of the upper eyelashes at the junction of the eyelash and the outer edge of the skin as shown in FIGS. 1 and 3-8. Next make sure to use a tissue to blot any excess solution beyond the lashes and dispose of the applicator. Avoid excess use of the solution, lest it may not come in contact with the cornea and conjunctiva. The bottles are containing therapeutic agents are supplied with multiple sterile applicators and instructions.

The side effect after application can be itching of the eyes and redness which will go away with continued use. Less common side effects are ocular irritation, dryness and redness of the eyelids. These symptoms occur on the eyelid where the product comes into contact with the skin and lid conjunctiva. Another side effect is hyper-pigmentation or darkening of the skin where the product like Latisse (Xaltan) is applied. Discontinue the treatment till it disappears and restart the program. Latisse needs a prescription from a doctor in order to purchase Latisse like you would get any other medication. Use of simple insulin to enhance the hair growth did not result in any appreciable darkening of the eye lids as noticed with prostaglandin preparations. There was no change in blood glucose levels after application of the insulin to the eye lids. If one develops hypoglycemic symptoms due to any number of reasons, drink a sugar drink, eat a candy, or fruit cocktail. We never have to use sugar to correct hypoglycemia because none of our cases developed hypoglycemia.

The Following Steps will Help to Use the Products of this Invention

Step 1: The eye and eyelids are cleared totally until the entire make-up and oily substances are removed. Then the eyelids are dried completely.

Step 2: Apply Oxidant to the affected area with a Q tip or triangular applicator provide with the medications of this invention. In this aspect, the oxidant may range from 2% to 10%, 2% to 9.5%, 3% to 8%, 3% to 7%, or 4% to 6% weight per volume, weight per weight, or by mole percent. In this aspect, the oxidant may range from 2% to 10%, 2% to 9.5%, 3% to 8%, 3% to 7%, or 4% to 6% weight per volume, weight per weight, or by mole percent. It is our observation, that using insulin on the eye lids edge may not need an oxidant for insulin and other hair growth products to be effective on the hair follicle and hair bulb. This is because, the hair roots are superficially place and there is easy access to the insulin and other therapeutic agents to get access to act on the hair follicle of the eyelashes to grow. Hence the step 2 is optional and may not be part of the example we describe below.

Step 3: Apply insulin by sterile Q tip or triangular applicator after the Oxidant (and/or permeability enhancer) are applied. Insulin can be in the form of watery dilute solution, gel or semi liquid or ointment can be applied without any of the absorption enhancers or oxidants.

Step 4: Allow the insulin to get absorbed and act on hair follicles for 15 minutes and then apply the selected hair growth factor (hypertrichotic effect) on the edge of both lids of both eyes and leave it to be absorbed. The application of this biochemical therapeutic agent with insulin can be repeated after 4-12 hours if rapid eyelash growth is desired.

Step 5: Optional step: Absorption enhancers, vasodilators, anti-inflammatory therapeutic agents can be applied if there is an underlying cause affecting the eyelid leading eyelash loss or noticeable slow growth.

Though we give an example of 5 step process of using our invention, the step can be summarized as follows:

Step 1: Clean the entire make-up and oily applications on the eyelids. Dry the eyelids completely at the site of therapeutic intervention

Step 2: Apply selected oxidant to the edge of the eyelid outside the hair line away from the conjunctiva sac and cornea. This step is optional and we have avoided it in many cases.

Step 3: Apply insulin by using sterile Q tip or triangular applicator. Repeatedly apply it as it dries out so that the cells of the hair follicle absorb it completely in high concentrations.

Step 4: Apply selected hair growth therapeutic agent besides insulin if desired or advised.

Step 5: Optional step: Absorption enhancers, vasodilators, anti-inflammatory therapeutic agents can be applied if there is a underlying cause affecting the eyelids.

These steps can be repeated 2-3 times a day if need be and best applied at bed time.

### Example 1

### Application of Insulin Alone as Hair Growth Factor without Other Therapeutic Agents

Step 1: Clean the entire make-up and oily applications on the eye lids. Dry the eyelids completely at the site of therapeutic intervention

Step 2: Apply oxidant to the edge of the eyelid. It is an optional step.

Step 3: Apply insulin by Q tip or triangular applicator. Repeatedly apply it as it dries out so that the cells of the hair follicle absorb it completely and get stimulated to multiply and grow to enhance the eyelash growth.

Step 4: No other hair growth enhancer is used.

Step 5: Optional step: Absorption enhancers, vasodilators, anti-inflammatory therapeutic agents can be applied if there is an underlying cause affecting the eyelids.

### Example 2

### Application of Insulin and Minoxidil

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth therapeutic agent. In this present example we choose to use Minoxidil liquid, gel or cream. This therapeutic agent is already available in foam form for application to the balding scalp. Pretreatment of insulin before application will enhance the hair growth much rapidly in a short duration. They hair that are growing from using our method are robust, resulting in dark and rapid growth.

Step 5: Optional step: Absorption enhancers, vasodilators, anti-inflammatory therapeutic agents is optional.

### Example 3

### Application of Insulin as Hair Growth Factor with Other Therapeutic Agents

Step 1: Clean the entire make-up and oily applications on the eyelids. Dry the eyelids completely at the site of therapeutic intervention.

Step 2: Apply oxidant to the edge of the eyelid to enhance the uptake of biologically active insulin to the eyelash roots. It is an optional step.

Step 3: Apply insulin by Q tip or triangular applicator. Repeatedly apply it as it dries out so that the cells of the hair follicle absorb it completely.

Step 4: Apply finasteride (Propecia) as hair growth enhancer is used.

Step 5: Optional step: Absorption enhancers, vasodilators, anti-inflammatory therapeutic agents can be applied if there is an underlying cause affecting the eyelids.

### Example 4

### Application of Insulin and P-1075, (Cromakalim), Diazoxide or Nicorandil

Step 1: The eye and eyelids are thoroughly cleaned till the entire make and up oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth therapeutic agent. In this present example we choose to use P-1075, (cromakalim), diazoxide or nicorandil instead of Minoxidil liquid, gel or cream. All these 3 therapeutic agents exert their hypertrichotic effect as potassium channel agonist in the vascular smooth muscles causing vasodilatation and enhanced blood supply to the hair papilla and follicle. Any and all available Potassium channel agonists can be used instead these three known available therapeutic agents.

Step 5: Optional step: Absorption enhancers, vasodilators, anti-inflammatory therapeutic agents is optional.

### Example 5

### Local Application of Insulin and Cyclosporine, and Hexachlorobenzene; Anabolic Steroids; Danazol

Step 1: The eye and eyelids are thoroughly cleaned till the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth therapeutic agent. In this present example we choose Cyproterone Acetate (CPA). It is steroidal androgen blocker. Taking oral forms will have feminizing effect in males, hence only ointment, gel or foam, liquid form needs to be formulated and used. Cyclosporine, and hexachlorobenzene, Anabolic steroids; Danazol can also be applied for haft growth locally.

Step 5: Optional step: Absorption enhancers, vasodilators, anti-inflammatory therapeutic agents is optional.

### Example 6

### Application of Insulin and Aromatase Inhibitors

Step 1: The eye and eyelids are thoroughly cleaned till the entire make and up oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth therapeutic agent. In this present example we choose to use aromatase inhibitors which work by inhibiting the action of the enzyme aromatase, which converts androgens into estrogens by a process called aromatization. As breast tissue is stimulated by estrogens, decreasing their production is a way of suppressing recurrence of the breast tumor tissue. There are numerous Aromatase inhibitors such as: Testolactone (Teslac), Anastrozole (Arimidex), Letrozole (Femara), Exemestane, (Aromasin), Vorozole (Rivizor), Formestane (Lentaron), Fadrozole (Afema), 4-androstene-3,6,17-trione (“6-OXO”, marketed as a nutritional supplement for athletes and weight lifters), 1,4,6-androstatrien-3,17-dione (ATD), 4-hydroxyandrostenedione, Aminoglutethimide, 4-OH androstenedione, 6-hydroximino-andmstenedione, 9,10-dimethyl 1,2-benzanthraccne, 1,4,6-androstatrien-3,17-dione, Atamestane, Exemstane, Fadrozolc, 1,2,4-triazole-3-alamine, Rogletimide and many are available in the market. We can select any one of the suitable therapeutic agent for local application after topical insulin application preparation of step 3. Natural Aromatase inhibitors: Quercetin, naringenin, resveratrol, apigenin, genistein, and oleuropein are all powerful flavonoids from whole foods that inhibit aromatase while at the same time offering a treasure chest of other health benefits can also be used for hair growth.

Step 5: Optional step: Absorption enhancers, vasodilators, anti-inflammatory therapeutic agents is optional.

### Example 7

### Application of Insulin and Growth Factors Known to Promote Growth of Hair

Step 1: The eye and eyelids are thoroughly cleaned till the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth therapeutic agent. It is another intent of this invention to add additional growth factors known to promote growth of hair; such factors include, without limitation, insulin, insulin-like growth factor (IGF-1), Relaxin, interleukin-4 (IL-4), transforming growth factor (TGF) (e.g., TGFa or TGFPI), basic fibroblast growth factor (bFGF), epidermal growth factor (EGF), platelet-derived growth factor (PDGF), or biotin.

Step 5: Optional step: Absorption enhancers, vasodilators, anti-inflammatory therapeutic agents is optional.

### Example 8

### Application of Insulin and Deferoxamine

Step 1: The eye and eye lids are thoroughly cleaned till the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth therapeutic agent such as Deferoxamine; also known as desferrioxamine B, desferoxamine B, DFO-B, DFOA, DFB or desferal. It is a bacterial siderophore produced by the actinobacter Streptomyces pilosus. It has medical applications as a chelating agent used to remove excess iron (and aluminum) from the body (Miller, Marvin J. 1989. “Syntheses and therapeutic potential of hydroxamic acid based siderophores and analogs”. Chemical Reviews 89 (7): 1563-1579). The mesylate salt of DFO-B is commercially available that can be applied on the eye lid margin. The dilute solution can also be injected subcutaneously to the eye lids in very small doses. Deferoxamine acts by binding free iron in the hair follicle location of papilla and remove to the bloodstream and enhancing its elimination in the urine. By removing excess iron, the agent reduces the damage done to various organs and tissues by iron mediated free radicals' damage and enhances angiogenesis'. Studies at UAB has shown that has angiogenic effect, hence can increase the blood supply to the hair papilla to promote hair growth. (http://www.sciencedaily.com/releases/2008/01/12.080110085148.htm).

A recent study also shows that it speeds healing of nerve damage (and minimizes the extent of recent nerve trauma) and bones and may modulate expression and release of inflammatory mediators by specific cell type (Lee H J, Lee J, Lee S K, Lee S K, Kim E C. Differential regulation of iron chelator-induced IL-8 synthesis via MAP kinase and NF-kappaB in immortalized and malignant oral keratinocytes. BMC Cancer. 2007 Sep. 13; 7:176. PMID: 17850672. Choi E Y, Kim E C, Oh H M, Kim S, Lee H J, Cho E Y, Yoon K H, Kim E A, Han W C, Choi S C, Hwang J Y, Park C, Oh B S, Kim Y, Kimm K C, Park K I, Chung H T, Jun C D. Iron chelator triggers inflammatory signals in human favourable to angiogenesis and removal free radicals).

Step 5: Optional step: Absorption enhancers, vasodilators, anti-inflammatory therapeutic agents is optional.

### Example 9

### Application of Insulin and Hair Follicle Transplant

One known treatment for male pattern alopecia is hair transplantation. Another expensive method of treatment of baldness is hair transplant in plugs or specially separated follicle injection to the balding scalp. Plugs of skin containing hair are transplanted from areas of the scalp where hair is growing to bald areas. The entire transplanted plug to do not take to start new hair growth. It is intent of this invention to facilitate the high percentage of transplanted hairs in the sub dermal area take root and also make them start growing as soon as the wound is begin to heal. The present invention describes the following method to achieve high percentage of successes and rapid growth of hair plugs or hair follicle transplant when used to grow new eye lashes which are totally shed and no hopes of getting them to grow by any known method using various therapeutic agents described in various inventions.

Step 1: The eye and eyelids of hair follicle site are thoroughly cleaned till the entire make-up and oily substances are removed. The eyelids are dried completely free of any oily substance.

Step 2: Apply Oxidant if need be, but it is optional. Povidine iodine acts as antiseptic also to prevent any infection of the transplanted site on the eyelids. It is an optional step.

Step 3: Apply fast acting insulin or IGF-1 by Q tip or triangular applicator after the Oxidant (and/or permeability enhancer) are applied. Insulin can be in the form of watery dilute solution, gel or semi liquid or ointment.

Step 4: Apply selected hair growth therapeutic agent if need be such as minoxidil or prostaglandin derived hair growth promoters which are already approved for hair growth of the eyelashes. In our experience, we do not need a second hypertrichosis therapeutic agent. Insulin is enough to facilitate rapid wound healing along with uptake and growth of hair follicle transplant resulting in healthy sprouting of hair follicles with rapid growth once the hair shaft emerge from the dermis and epidermis. Apply the clean dressing on the wound if need be.

Step 5: Optional step: Absorption enhancers, vasodilators, anti-inflammatory therapeutic agents is optional.

### Example 10

### Application of Insulin and Prostaglandins and Derivatives

Step 1: The eye and eyelids are thoroughly cleaned till the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth therapeutic agent, which includes prostaglandin and its derivatives which are already in the market and FDA approved. Prostaglandins and derivatives thereof are useful in a method of enhancing hair growth. Application of the hair growth promoting prostaglandins to accelerate the robust eyelashes hair growth compared to when used without insulin.

Step 5: Optional step: Absorption enhancers, vasodilators, anti-inflammatory therapeutic agents is optional.

### Example 11

### Application of Insulin and Extracts of Cotinus coggygria

Step 1: The eye and eyelids are thoroughly cleaned till the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply extracts of Cotinus coggygria; a selected hair growth and hair quality improving agent in an amount effective to induce hair growth when applied topically to an area of the eyelid skin on which hair growth is desired.

Step 5: Optional step: Absorption enhancers, vasodilators, anti-inflammatory therapeutic agents is optional.

### Example 12

### Application of Insulin and Use of a Device Relating to Effective Delivery by Targeted Application of Therapeutically Effective Hair Growth Agents to the Eye Lashes

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Use a device that targets a specific site on the eyelashes which helps limit and/or eliminate concerns about the cosmeceuticals active ingredient effecting unintended areas of the body.

Step 5: Optional step: Absorption enhancers, vasodilators, anti-inflammatory therapeutic agents is optional.

### Example 13

### Application of Insulin and Photodynamic Therapy (PDT)

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Using a photodynamic therapy (PDT) to stimulate an increase in hair count numbers and restore hair growth in areas of hair loss. A method according to the present invention comprises:

a) administering an effective and/or sufficient amount of a photosensitizer to the target skin; the insulin priming will allow the photosensitizer to be distributed at the site of dermal papilla evenly;

b) irradiating the target skin with energy comprising one or more wavelength capable of activating said photosensitizer for a time period sufficient to in the treated area. In one aspect of the present invention, there is a 2% or more increase in the number of terminal hairs within 3 months. This method may be difficult to be adapted to growth of eyelashes. If used, pretreatment with insulin will enhance the effectiveness of the PDT.

Step 5: Optional step: Absorption enhancers, vasodilators, anti-inflammatory therapeutic agents are optional and may not be needed when the PDT is used.

### Example 14

### Application of Insulin and Local Application of Tocotrienols

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Tocotrienol is a form of vitamin E. It is a potent anti-oxidant and has been used in combating many health problems. There was a report of the beneficial effects of vitamin E in hair care products (Shipp J Jesus; 1994. Hair care products. In: Chemistry and technology of the cosmetics and toiletries industry. Williams D F and Schmitt W H, ed.s), p 66. Blackie Academic & Professional: UK.). U.S. PATENT APPLICATION PUB. NO.: 200410009135 A1 discloses the local application of tocotrienols. We recommend the use of tocotrienols after insulin application so that this therapeutic agent can easily permeate the hair eyelid hair line and promote the eyelash hair growth. Another antioxidant we have used effectively is and extract of turmeric called curcumin.

Step 5: Optional step: Absorption enhancers, vasodilators, anti-inflammatory therapeutic agents is optional.

### Example 15

### Application of Insulin and Use Hair Follicle with Adipose Tissue to be Implanted to the Balding Area

Step 1: The eye and eyelids are thoroughly cleaned till the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: The roots of actively growing hairs are embedded in a layer of fat cells (adipocytes). Application of insulin post surgical make the hair root take the scalp and emerge as terminal hair lasting a life time.

Step 5: Optional step: Absorption enhancers, vasodilators, anti-inflammatory therapeutic agents is optional.

### Example 16

### Application of Insulin and a 5a-Reductase Inhibitor

Step 1: The eye and eye lids are thoroughly cleaned till the entire make and up oily substances are removed. The eye lids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply a selected hair growth therapeutic agent such as a hair growth stimulant comprising p-menthane-3,8-diol and at least one substance from the group of a blood circulation promoter, a 5a-reductase inhibitor (Examples of the 5-a-reductase inhibitor P-glycyrrhetic acid, estradiol and estrone), an antihistaminic (diphenhydramine hydrochloride), a cell activator (hair follicular function include pantothenyl alcohol, pantothenyl ethyl ether, Photosensitizer 301 and extract of ginseng); those of the antiphlogistic include glycynhetic acid and derivatives those of the antimicrobial include hinokitiol and isopropyl methyl phenol in combination. The hair growth stimulant is free from hormonal action, does not cause side effects and has excellent hair growth promoting effects.

Step 5: Optional step: Absorption enhancers, vasodilators, anti-inflammatory therapeutic agents is optional.

### Example 17

### Application of Insulin and an Agent for Hair Growth Agent Comprising a Processed Semi-Mature Soybean and/or a Processed Semi-Mature Soybean Extracts

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply a selected hair growth therapeutic agent characterized by using a processed semi-mature soybean and/or a processed semi-mature soybean extracts and at least one substance selected from the group consisting of a processed Polygoni Multiflori Radix, processed Polygoni Multiflori Radix extracts, a processed Cynanchum bungei Decne or processed Cynanchum bungei Decne extracts, preferably further comprising Longan seed and or Longan seed extracts as active ingredients.

This agent for hair growth has no side effects when used externally or internally, it can notably improve hair growth within a short period of time; ranging from 6 to 12 weeks, can return hair to its normal hair color (for example from white to black) and can improve the gloss of hair. This hair tonic can be more effective using our invention of preparing the site of hair loss with insulin and then apply the products described in this invention.

Step 5: Optional step: Absorption enhancers, vasodilators, anti-inflammatory therapeutic agents is optional.

### Example 18

### Application of Insulin and an Ethanol or Aqueous Ethanol Preparation of Fatty Acid, Fatty Acid Ester, Polyglycerin Fatty Acid Esters and at Least One of Sorbitan Fatty Acid Esters

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply a selected hair growth therapeutic which is an ethanol or aqueous ethanol preparation comprising, as active ingredients for a hair growth promoter, (A) at least one compound selected from fatty acids having a chain length of an odd number of carbon atoms, the derivatives of the fatty acids, aliphatic alcohols having a chain length of an odd number of carbon atoms and the derivatives of the aliphatic alcohols and (B) at least one selected from 6-benzylaminopurine and/or the derivatives thereof represented by the following Formula (I), wherein it further comprises (C) at least one of polyglycerin fatty acid esters and at least one of sorbitan fatty acid esters: in Formula (I), R, and R, are defined. The hair growth promoter can have a good hair growth effect and can provide an excellent stabilization effect at low temperature and can provide good feeling having no stickiness. Use of it after insulin application can enhance the effectiveness of above therapeutic agents.

Step 5: Optional step: Absorption enhancers, vasodilators, anti-inflammatory therapeutic agents is optional.

### Example 19

### Application of Insulin and a Vascular Endothelial Growth Factor (VEGF)

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth therapeutic agent using modulating VEGF (vascular endothelial growth factor) activity, e.g., modulating VEGF gene expression and/or modulating VEGF protein production and/or activity, to modulate hair growth and/or thickness. The methods can be used to either promote or inhibit hair growth or hair thickness in a subject. Use of VEGR increased perifollicular vascularization promotes hair growth and that expression of VEGF by follicular keratinocytes leads to increased perifollicular vascularization. In addition, it was found that increasing the level of VEGF expression resulted in accelerated hair regrowth and increased hair follicle size, which leads to hair thickening. It was also found that by inhibiting the levels of VEGF, hair growth and hair thickening can be reduced. Applying the VEGF after step 3 insulin application will enhance the activity of VEGF and will lead to rapid hair growth. Insulin itself acts as and enhances the activity of VEGF.

Step 5: Optional step: Absorption enhancers, vasodilators, anti-inflammatory therapeutic agents is optional.

### Example 20

### Application of Insulin and a Therapeutic Agent Inhibiting 5α-Reductase Activity and an Ingredient for Dilating Peripheral Blood Vessels

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth promoting therapeutic agent using:

a) an ingredient for inhibiting 5 a-reductase activity;

b) an ingredient for promoting function of cell activity; and

c) an ingredient for dilating peripheral blood vessels

Step 5: Optional step: Absorption enhancers, vasodilators, anti-inflammatory therapeutic agents is optional.

### Example 21

### Application of Insulin and Therapeutic Agents Comprising PGF 2α, and Analogues

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth therapeutic agent using PGF 2α, and analogues. Using these hair growing prostaglandins after pretreatment of the eye lids site with insulin results in much more rapid hair growth compared to when applied without insulin.

Step 5: Optional step: Absorption enhancers, vasodilators, anti-inflammatory therapeutic agents is optional.

### Example 22

### Application of Insulin and a Compound Having a Cholinesterase Inhibiting Action and Has Hair Growth Promoting Action

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Then apply selected hair growth promoting therapeutic agent using a compound having a cholinesterase inhibiting action has a hair growth promoting action, and there is provided a hair growth promoting agent containing such a compound having a cholinesterase inhibiting action. Moreover, a compound having an acetylcholinesterase inhibiting action as the cholinesterase inhibiting action is preferable, and in particular donepezil hydrochloride has a good hair growth promoting effect applied as liquid preparation, a cream, an ointment, a plaster or a tape preparation. Moreover, according to the present invention, there is also provided a hair growth promoting method comprising the step of applying donepezil onto the eye lids after applying insulin to enhance the uptake, activity and spread of this hair growth promoter.

Step 5: Optional step: Absorption enhancers, vasodilators, anti-inflammatory therapeutic agents is optional.

### Example 23

### Application of Insulin and Use of Testosterone and Dihyrdotestosterone, Potassium Channel Openers as Described above, Streptomycin, Diphenylhydantoin

Step 1: The eye and eyelids are thoroughly cleaned until the entire make-up and oily substances are removed. The eyelids are dried completely free of any oily substance.

Step 2: Apply Oxidant to the affected area with a Q tip or triangular applicator provide with the medications of this invention. It is an optional step.

Step 3: Apply fast acting insulin by Q tip or triangular applicator after the Oxidant (and/or permeability enhancer) are applied. Insulin can be in the form of watery dilute solution, gel or semi liquid or ointment.

Step 4: Apply selected hair growth therapeutic agent other than aromatase inhibitors, including testosterone and dihyrdotestosterone, potassium channel openers as described above, streptomycin, diphenylhydantoin (anticonvulsant drug widely used to control epileptic seizures).

Step 5: Optional step: Absorption enhancers, vasodilators, anti-inflammatory therapeutic agents is optional.

### Example 24

### Application of Insulin Alone as Hair Growth Factor without Other Therapeutic Agents

Step 1: Clean the entire make-up and oily applications on the eyelids. Dry the eyelids completely at the site of therapeutic intervention

Step 2: Apply no oxidant to the edge of the eyelid. It is an optional step.

Step 3: Apply insulin growth factor (IGF-1) with sterile Q tip or triangular applicator. Repeatedly apply it as it dries out so that the cells of the hair follicle absorb it completely and get stimulated to multiply and grow to enhance the eye lash growth.

Step 4: May or may not use other hair growth enhancer discussed here in following the application of IGR-1.

Step 5: Optional step: Absorption enhancers, vasodilators, anti-inflammatory therapeutic agents can be applied if there is an underlying cause affecting the eye lids.

All the above described examples can also be used to treat eyebrow hair loss or to enhance their growth to restore the normal aesthetic appearance.

Numerous modifications and alternative arrangements of steps explained herein may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form function and manner of procedure, assembly and use may be made.

While the preferred embodiment of the present invention has been described, it should be understood that various changes, adaptations and modifications may be made thereto. It should be understood, therefore, that the invention is not limited to details of the illustrated invention.

1. A method of increasing the length, thickness, number, and density of eyelash hair and eyebrow hair, comprising the step of administering a therapeutically effective amount of insulin to a person on an area where hair growth is desired.

2. (canceled)

3. The method of increasing the length, thickness, number, and density of eyelash hair and eyebrow hair according to claim 1 further comprising the step of applying a known hair growth therapeutic agent to said area.

4-17. (canceled)
